SEQUENCE LISTING (113 sequences)

SEQ ID NO: 1 - protein, 30 aa, Homo sapiens
MKTLPAMLGTGKLFWVFFLIPYLDIWNIHG

SEQ ID NO: 2 - protein, 20 aa, Homo sapiens
KESCDVQLYIKRQSEHSILA

SEQ ID NO: 3 - protein, 65 aa, Homo sapiens
GDPFELECPVKYCANRPHVTWCKLNGTTCVKLEDRQTSWKEEKNISFFILHFEPVLPNDNGSYRC

SEQ ID NO: 4 - protein, 42 aa, Homo sapiens
SANFQSNLIESHSTTLYVTDVKSASERPSKDEMASRPWLLYS

SEQ ID NO: 5 - protein, 21 aa, Homo sapiens
LLPLGGLPLLITTCFCLFCCL

SEQ ID NO: 6 - protein, 44 aa, Homo sapiens
RRHQGKQNELSDTAGREINLVDAHLKSEQTEASTRQNSQVLLSE

SEQ ID NO: 7 - protein, 8 aa, Homo sapiens
TGIYDNDP

SEQ ID NO: 8 - protein, 23 aa, Homo sapiens
DLCFRMQEGSEVYSNPCLEENKP

SEQ ID NO: 9 - protein, 9 aa, Homo sapiens
GIVYASLNH

SEQ ID NO: 10 - protein, 14 aa, Homo sapiens
SVIGLNSRLARNVK

SEQ ID NO: 11 - protein, 13 aa, Homo sapiens
EAPTEYASICVRS

SEQ ID NO: 12 - protein, 127 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYS

SEQ ID NO: 13 - protein, 157 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYS

SEQ ID NO: 14 - protein, 148 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCL

SEQ ID NO: 15 - protein, 178 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCL

SEQ ID NO: 16 - protein, 67 aa, Homo sapiens
TGIYDNDPDLCFRMQEGSEVYSNPCLEENKPGIVYASLNHSVIGLNSRLARNVKEAPTEYASICVRS

SEQ ID NO: 17 - protein, 111 aa, Homo sapiens
RRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 18 - protein, 132 aa, Homo sapiens
LLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 19 - protein, 239 aa, Homo sapiens
GDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 20 - protein, 259 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 21 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 22 - DNA, 90 bp, Homo sapiens
atgaagacattgcctgccatgcttggaactgggaaattattttgggtcttcttcttaatcccatatctggacatctggaacatccatggg

SEQ ID NO: 23 - DNA, 60 bp, Homo sapiens
aaagaatcatgtgatgtacagctttatataaagagacaatctgaacactccatcttagca

SEQ ID NO: 24 - DNA, 201 bp, Homo sapiens
ggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgca

SEQ ID NO: 25 - DNA, 120 bp, Homo sapiens
aattttcagtctaatctcattgaaagccactcaacaactctttatgtgacagatgtaaaaagtgcctcagaacgaccctccaaggacgaaatggcaagcagaccctggctcctgtatagt

SEQ ID NO: 26 - DNA, 63 bp, Homo sapiens
ttacttcctttggggggattgcctctactcatcactacctgtttctgcctgttctgctgcctg

SEQ ID NO: 27 - DNA, 132 bp, Homo sapiens
agaaggcaccaaggaaagcaaaatgaactctctgacacagcaggaagggaaattaacctggttgatgctcaccttaagagtgagcaaacagaagcaagcaccaggcaaaattcccaagtactgctatcagaa

SEQ ID NO: 28 - DNA, 24 bp, Homo sapiens
actggaatttatgataatgaccct

SEQ ID NO: 29 - DNA, 69 bp, Homo sapiens
gacctttgtttcaggatgcaggaagggtctgaagtttattctaatccatgcctggaagaaaacaaacca

SEQ ID NO: 30 - DNA, 27 bp, Homo sapiens
ggcattgtttatgcttccctgaaccat

SEQ ID NO: 31 - DNA, 42 bp, Homo sapiens
tctgtcattggactgaactcaagactggcaagaaatgtaaaa

SEQ ID NO: 32 - DNA, 39 bp, Homo sapiens
gaagcaccaacagaatatgcatccatatgtgtgaggagt

SEQ ID NO: 33 - DNA, 381 bp, Homo sapiens
aaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagt

SEQ ID NO: 34 - DNA, 471 bp, Homo sapiens
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagt

SEQ ID NO: 35 - DNA, 444 bp, Homo sapiens
aaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctg

SEQ ID NO: 36 - DNA, 534 bp, Homo sapiens
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctg

SEQ ID NO: 37 - DNA, 201 bp, Homo sapiens
actggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 38 - DNA, 333 bp, Homo sapiens
agaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 39 - DNA, 396 bp, Homo sapiens
ttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 40 - DNA, 717 bp, Homo sapiens
ggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 41 - DNA, 777 bp, Homo sapiens
aaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagt

SEQ ID NO: 42 - DNA, 870 bp, Homo sapiens
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagttaa

SEQ ID NO: 43 - protein, 29 aa, Mus musculus
MKTVPAMLGTPRLFREFFILHLGLWSILC

SEQ ID NO: 44 - protein, 27 aa, Mus musculus
EKATKRNDEECPVQLTITRNSKQSART

SEQ ID NO: 45 - protein, 68 aa, Mus musculus
GELFKIQCPVKYCVHRPNVTWCKHNGTICVPLEVSPQLYTSWEENQSVPVFVLHFKPIHLSDNGSYSC

SEQ ID NO: 46 - protein, 56 aa, Mus musculus
STNFNSQVINSHSVTIHVTERTQNSSEHPLITVSDIPDATNASGPSTMEERPGRTW

SEQ ID NO: 47 - protein, 24 aa, Mus musculus
LLYTLLPLGALLLLLACVCLLCFL

SEQ ID NO: 48 - protein, 37 aa, Mus musculus
KRIQGKEKKPSDLAGRDTNLVDIPASSRTNHQALPSG

SEQ ID NO: 49 - protein, 8 aa, Mus musculus
TGIYDNDP

SEQ ID NO: 50 - protein, 21 aa, Mus musculus
WSSMQDESELTISLQSERNNQ

SEQ ID NO: 51 - protein, 9 aa, Mus musculus
GIVYASLNH

SEQ ID NO: 52 - protein, 14 aa, Mus musculus
CVIGRNPRQENNMQ

SEQ ID NO: 53 - protein, 13 aa, Mus musculus
EAPTEYASICVRS

SEQ ID NO: 54 - protein, 151 aa, Mus musculus
EKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTW

SEQ ID NO: 55 - protein, 180 aa, Mus musculus
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTW

SEQ ID NO: 56 - protein, 175 aa, Mus musculus
EKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFL

SEQ ID NO: 57 - protein, 204 aa, Mus musculus
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFL

SEQ ID NO: 58 - protein, 65 aa, Mus musculus
TGIYDNDPWSSMQDESELTISLQSERNNQGIVYASLNHCVIGRNPRQENNMQEAPTEYASICVRS

SEQ ID NO: 59 - protein, 102 aa, Mus musculus
KRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 60 - protein, 126 aa, Mus musculus
LLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 61 - protein, 250 aa, Mus musculus
GELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 62 - protein, 277 aa, Mus musculus
EKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 63 - protein, 306 aa, Mus musculus
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 64 - DNA, 87 bp, Mus musculus
atgaagacagtgcctgccatgcttgggactcctcggttatttagggaattcttcatcctccatctgggcctctggagcatcctttgt

SEQ ID NO: 65 - DNA, 81 bp, Mus musculus
gagaaagctactaagaggaatgatgaagagtgtccagtgcaacttactattacgaggaattccaaacagtctgccaggaca

SEQ ID NO: 66 - DNA, 210 bp, Mus musculus
ggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctaca

SEQ ID NO: 67 - DNA, 162 bp, Mus musculus
aacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttgg

SEQ ID NO: 68 - DNA, 72 bp, Mus musculus
ctgctttacaccttgcttcctttgggggcattgcttctgctccttgcctgtgtctgcctgctctgctttctg

SEQ ID NO: 69 - DNA, 111 bp, Mus musculus
aaaaggatccaagggaaagaaaagaagccttctgacttggcaggaagggacactaacctggttgatattccagccagttccaggacaaatcaccaagcactgccatcagga

SEQ ID NO: 70 - DNA, 24 bp, Mus musculus
actggaatttatgataatgatccc

SEQ ID NO: 71 - DNA, 63 bp, Mus musculus
tggtctagcatgcaggatgaatctgaattgacaattagcttgcaatcagagagaaacaaccag

SEQ ID NO: 72 - DNA, 27 bp, Mus musculus
ggcattgtttatgcttctttgaaccat

SEQ ID NO: 73 - DNA, 42 bp, Mus musculus
tgtgttattggaaggaatccaagacaggaaaacaacatgcag

SEQ ID NO: 74 - DNA, 39 bp, Mus musculus
gaggcacccacagaatatgcatccatttgtgtgagaagt

SEQ ID NO: 75 - DNA, 453 bp, Mus musculus
gagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttgg

SEQ ID NO: 76 - DNA, 540 bp, Mus musculus
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttgg

SEQ ID NO: 77 - DNA, 525 bp, Mus musculus
gagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctg

SEQ ID NO: 78 - DNA, 612 bp, Mus musculus
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctg

SEQ ID NO: 79 - DNA, 195 bp, Mus musculus
actggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 80 - DNA, 306 bp, Mus musculus
aaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 81 - DNA, 378 bp, Mus musculus
ctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 82 - DNA, 750 bp, Mus musculus
ggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 83 - DNA, 831 bp, Mus musculus
gagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagt

SEQ ID NO: 84 - DNA, 921 bp, Mus musculus
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaa

SEQ ID NO: 85 - protein, 179 aa, Mus musculus
MKTVPAMLGTPRLFREFFILHLGLWSILCEKATKRNDEECEVQLNIKRNSKHSAWTGELFKIECPVKYCVHRPHVTWCKHNGTIWVPLEVGPQLYTSWEENRSVPVFVLHFKPIHLSDNGSYSCSTNFNSQVINSHSVTIHVRERTQNSSEHPLITVSDIPDATNASGPSTMEERPGRT

SEQ ID NO: 86 - protein, 150 aa, Mus musculus
EKATKRNDEECEVQLNIKRNSKHSAWTGELFKIECPVKYCVHRPHVTWCKHNGTIWVPLEVGPQLYTSWEENRSVPVFVLHFKPIHLSDNGSYSCSTNFNSQVINSHSVTIHVRERTQNSSEHPLITVSDIPDATNASGPSTMEERPGRT

SEQ ID NO: 87 - protein, 29 aa, Mus musculus
MKTVPAMLGTPRLFREFFILHLGLWSILC

SEQ ID NO: 88 - protein, 68 aa, Mus musculus
GELFKIECPVKYCVHRPHVTWCKHNGTIWVPLEVGPQLYTSWEENRSVPVFVLHFKPIHLSDNGSYSC

SEQ ID NO: 89 - DNA, 537 bp, Mus musculus
atgaagacagtgcctgccatgcttgggactcctcggttatttagggaattcttcatcctccatctgggcctctggagcatcctttgtgagaaagctactaagaggaatgatgaagagtgtgaagtgcaacttaatattaagaggaattccaaacactctgcctggacaggagagttatttaaaattgaatgtcctgtgaaatactgtgttcatagacctcatgtgacttggtgtaagcacaatggaacaatctgggtaccccttgaagttggtcctcagctatacactagttgggaagaaaatcgatcagttccggtttttgttctccattttaaaccaatacatctcagtgataacgggtcgtatagctgttctacaaacttcaattctcaagttattaatagccattcagtaaccatccatgtgagagaaaggactcaaaactcttcagaacacccactaataacagtatctgacatcccagatgccaccaatgcctcaggaccatccaccatggaagagaggccaggcaggact

SEQ ID NO: 90 - DNA, 450 bp, Mus musculus
gagaaagctactaagaggaatgatgaagagtgtgaagtgcaacttaatattaagaggaattccaaacactctgcctggacaggagagttatttaaaattgaatgtcctgtgaaatactgtgttcatagacctcatgtgacttggtgtaagcacaatggaacaatctgggtaccccttgaagttggtcctcagctatacactagttgggaagaaaatcgatcagttccggtttttgttctccattttaaaccaatacatctcagtgataacgggtcgtatagctgttctacaaacttcaattctcaagttattaatagccattcagtaaccatccatgtgagagaaaggactcaaaactcttcagaacacccactaataacagtatctgacatcccagatgccaccaatgcctcaggaccatccaccatggaagagaggccaggcaggact

SEQ ID NO: 91 - DNA, 87 bp, Mus musculus; identical to SEQ ID NO: 64

SEQ ID NO: 92 - DNA, 204 bp, Mus musculus
ggagagttatttaaaattgaatgtcctgtgaaatactgtgttcatagacctcatgtgacttggtgtaagcacaatggaacaatctgggtaccccttgaagttggtcctcagctatacactagttgggaagaaaatcgatcagttccggtttttgttctccattttaaaccaatacatctcagtgataacgggtcgtatagctgt

SEQ ID NO: 93 - protein, 6 aa, Artificial Sequence
  Xaa = any amino acid
IXYXXL

SEQ ID NO: 94 - protein, 6 aa, Artificial Sequence
  Synthesized
IVYASL

SEQ ID NO: 95 - protein, 6 aa, Artificial Sequence
  Xaa = any amino acid
TXYXXI

SEQ ID NO: 96 - protein, 6 aa, Artificial Sequence
  Synthesized
TEYASI

SEQ ID NO: 97 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 521 aa, Artificial Sequence
  Fusion polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGLNSRLARNVKEAPTEYASICVRS

SEQ ID NO: 99 - protein, 538 aa, Artificial Sequence
  Fusion polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 100 - protein, 411 aa, Artificial Sequence
  Fusion polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMKTVPAMLGTPRLFREFFILHLGLWSILCEKATKRNDEECEVQLNIKRNSKHSAWTGELFKIECPVKYCVHRPHVTWCKHNGTIWVPLEVGPQLYTSWEENRSVPVFVLHFKPIHLSDNGSYSCSTNFNSQVINSHSVTIHVRERTQNSSEHPLITVSDIPDATNASGPSTMEERPGRT

SEQ ID NO: 101 - protein, 8 aa, Artificial Sequence
  Synthesized
DYKDDDDK

SEQ ID NO: 102 - protein, 10 aa, Artificial Sequence
  Synthesized
EQKLISEEDL

SEQ ID NO: 103 - protein, 7 aa, Artificial Sequence
  Synthesized
TGIYDND

SEQ ID NO: 104 - DNA, 990 bp, Homo sapiens
actggggtaggtaaactgacccaactctgcagcactcagaagacgaagcaaagccttctacttgagcagtttttccatcactgatatgtgcaggaaatgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatagtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggactgaactcaagactggcaagaaatgtaaaagaagcaccaacagaatatgcatccatatgtgtgaggagttaagtctgttttctgacctcccaacag

SEQ ID NO: 105 - DNA, 1250 bp, Mus musculus
agatctctagggaggaagaggaagtttgcccttacctgacacgtgctgggaatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaaacctgccactgagccaggcagcctacactgcatgagtgcctgtcaatacctctgtctggaccttcagtttcaaataaccttcaacctggaaagtttcaattaagatgctctgtgctggtgctgcgtcttaaaggtccatgaagtatttagttaaaatttctcctgaaaactttgggagagttttgtacaagacagactcttccctgggaaaactcatattacgaatagcagaataatagggcttttaaaattgaccatgtcgcaccatgtcgtaccca

SEQ ID NO: 106 - DNA, 957 bp, Mus musculus
ggcccgggatctatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagtgcagatccaccggtcgccaccatggtg

SEQ ID NO: 107 - DNA, 1722 bp, Mus musculus
ggatctcgagctcaagcttcgaattctgcagtcgacggtaccgcgggcccgggatctatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagtgcagatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggc

SEQ ID NO: 108 - DNA, 609 bp, Mus musculus
ggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaaacctgccactgagccaggcagcctacactgcatgagtgcctgtcaatacctctgtctggaccttcagtttcaaataaccttcaacctggaaagtttcaattaagatgctctgtgctggtgctgcgtcttaaaggtccatgaagtatttagttaaaatttctcctgaaaactttgggagagttttgtacaagacagactcttccctgggaaaactcatattacgaatagcagaataatagggcttttaaaattgaccatgtcgcaccatgtcgtacccaaagggcgaattctgcagatcagatct

SEQ ID NO: 109 - DNA, 1276 bp, Mus musculus
agatctctagggaggaagaggaagtttgcccttacctgacacgtgctgggaatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaaacctgccactgagccaggcagcctacactgcatgagtgcctgtcaatacctctgtctggaccttcagtttcaaataaccttcaacctggaaagtttcaattaagatgctctgtgctggtgctgcgtcttaaaggtccatgaagtatttagttaaaatttctcctgaaaactttgggagagttttgtacaagacagactcttccctgggaaaactcatattacgaatagcagaataatagggcttttaaaattgaccatgtcgcaccatgtcgtacccaaagggcgaattctgcagatcagatct

SEQ ID NO: 110 - DNA, 486 bp, Mus musculus
ggatcttggagaaagctactaagaggaatggagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggactagatcc

SEQ ID NO: 111 - DNA, 464 bp, Mus musculus
agatcttggagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggactagatct

SEQ ID NO: 112 - DNA, 538 bp, Mus musculus
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataacagtatctgacatcccagatgccaccaatgcctcaggaccatccaccatggaagagaggccaggcaggactgc

SEQ ID NO: 113 - DNA, 537 bp, Mus musculus; identical to SEQ ID NO: 89

(19) United States
(12) Patent Application Publication
(10) Pub. No.: US 2005/0152893 A1
(43) Pub. Date: Jul. 14, 2005

(54) Methods and materials for the inhibition of transplant rejection

(75) Inventor: Jonathan Kaye, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 10/964,215

(22) Filed: Oct. 12, 2004

Related U.S. Application Data
(63) Continuation-in-part of application No. 10/831,622, filed on Apr. 23, 2004.
(60) Provisional application No. 60/467,206, filed on Apr. 30, 2003.

(51) Int. Cl. A61K 39/395; A61K 38/13; A61K 31/4745; A61K 31/573

(52) U.S. Cl. 424/131.1; 514/11; 514/179; 514/291

(57) ABSTRACT

The present invention provides compositions and methods of inhibiting transplant rejection in a mammal, comprising administering one or more transplant rejection inhibiting compounds to the mammal, wherein at least one compound includes an antibody that immunoreacts with the extracellular domain of the SPEX polypeptide. The present invention also provides a method of decreasing an expression of the SPEX polypeptide on a lymphocyte that expresses the SPEX polypeptide. The present invention further provides a method of identifying antibodies that immunoreact with the extracellular domain of the SPEX polypeptide and inhibit transplant rejection.

### CROSS REFERENCE TO RELATED APPLICATION

The present application claims priority to U.S. application Ser. No. 10/831,622 filed Apr. 23, 2004, pending, which is a continuation-in-part of U.S. Provisional Application Ser. No. 60/467,206 filed Apr. 30, 2003, now abandoned.

### GOVERNMENT SUPPORT CLAUSE

This invention was made with government support under Contract A131231, awarded by the National Institutes of Health. The United States Government has certain rights in the invention.

### BACKGROUND OF THE INVENTION

1. Field of the Invention

The present invention is related to transplant rejection. In particular, this invention provides methods and compositions useful for inhibiting transplant rejection.

2. State of the Art

Current strategies for the treatment of graft rejection after transplantation typically involve the use of immunosuppressive agents such as cyclosporin A (CsA), rapamycin, FK506, corticosteriods, and antibodies to the interleukin (IL)-2 receptor. These drugs are typically taken over a long period of time, result in the global depletion of lymphocytes, and increase the risk of serious infection, nephrotoxicity, and cancer. Furthermore, some patients cannot tolerate doses of these immunosuppressive agents which are sufficient to inhibit transplant rejection.

What is needed are additional inhibitors of transplantation rejection, especially those that do not globally deplete lymphocytes from the individual receiving the inhibitor.

### SUMMARY OF THE INVENTION

One embodiment of the present invention provides a method of inhibiting transplant rejection in a mammal in need thereof, comprising: administering a therapeutically effective amount of an antibody to the mammal, wherein the antibody immunoreacts with an extracellular domain of a polypeptide referred to herein as “spleen expressed (SPEX) polypeptide”. In certain embodiments, the present method further comprises administering one or more additional transplant rejection inhibitor(s) to the mammal. Examples of additional transplant inhibitors include: CsA, rapamycin, FK506, corticosteriods, and antibodies to the IL-2 receptor.

One benefit of using the present anti-SPEX antibody in the inhibition of transplant rejection is that administration of the anti-SPEX antibody to mammals, including a transplant recipient, does not globally deplete lymphocytes in the mammal. Accordingly, one embodiment of the present invention provides a method of inhibiting transplant rejection in a mammal in need thereof, comprising: administering a therapeutically effective amount of an antibody to the mammal, wherein the antibody immunoreacts with an extracellular domain of a SPEX polypeptide, and wherein the administration of the antibody does not significantly decrease a total CD4⁺T cell count, a total CD8⁺T cell count, or a total B cell count in the mammal.

One embodiment of the present invention provides a method of decreasing an expression of a spleen expressed (SPEX) polypeptide on a lymphocyte that expresses the SPEX polypeptide, comprising contacting the lymphocyte with an anti-SPEX antibody that immunoreacts with an extracellular domain of a SPEX polypeptide, thereby decreasing the expression of the SPEX polypeptide on the lymphocyte.

One embodiment of the present invention provides a method of identifying an antibody that inhibits transplant rejection in a mammal in need thereof, comprising: providing an amino acid sequence comprising an epitope of an extracellular domain of a SPEX polypeptide, wherein said epitope is at least six amino acids in length; producing antibodies to said amino acid sequence; and screening said antibodies in a model of transplant rejection, thereby identifying said antibody that inhibits transplant rejection.

### LISTING OF THE FIGURES

The drawings form a portion of the specification of the present invention.

FIG. 1 provides a diagram of substantially full-length mouse and human SPEX polypeptides (mSPEX and hSPEX, respectively) having extracellular, transmembrane (crosshatched), and intracellular domains (as labeled). The extracellular domains each include an immunoglobulin (Ig) like domain (horizontally hatched) and optionally have a cleavable signal sequence (dashed areas), wherein the arrows point to the cleavage site. The signal sequence is cleaved during post-translational processing to generate the mature SPEX polypeptide. The intracellular domains each include three tyrosine based motifs (vertically hatched). Each tyrosine based domain, in turn includes a tyrosine amino acid residue (Y, is the one-letter code for tyrosine). The embodiments shown depict that the tyrosine can be optionally phosphorylated (encircled P). The relative position of the SPEX polypeptides in a biological membrane is shown. The stippled areas indicate portions of each polypeptide between the certain functional domains.

FIG. 2A provides a diagram showing embodiments of the correspondence between selected hSPEX polypeptides and the respective sequence identifier for each selected polypeptide.

FIG. 2B provides a diagram showing embodiments of the correspondence between selected hSPEX polynucleotides and the respective sequence identifier for each selected polynucleotide.

FIG. 2C provides a diagram showing certain embodiments of the correspondence between selected mSPEX polypeptides and the respective sequence identifier for each selected polypeptide.

FIG. 2D provides a diagram showing certain embodiments of the correspondence between selected mSPEX polynucleotides and the respective sequence identifier for each selected polynucleotide.

FIG. 3 shows syngeneic and allogeneic skin grafts on a control mouse (left panel) at day 9 post transplantation and on a PJ19.1 monoclonal antibody treated mouse (right panel) at day 13 post transplantation as described in Example 7. The control mouse was injected with PBS on days −1, 1, 4, and 6 relative to transplantation of the skin grafts. The PJ19.1 monoclonal antibody treated mouse was injected i.p. (intraperitoneal) with 500 μg of the antibody on −1, 1, 4, and 6 relative to transplantation of the skin grafts. The PJ19.1 monoclonal antibody immunoreacts with the extracellular domain of the mouse SPEX (mSPEX) polypeptide. The control mouse rejected the allogeneic graft by day 9, while maintaining the syngeneic graft. The PJ19.1 treated mouse maintains both the syngeneic and the allogeneic skin grafts at day 13.

FIG. 4 shows skin graft survival curves for allogeneic skin grafts used as a model system for transplant rejection as described in Example 7. The control mice (square symbols) received syngeneic (data not shown) and allogeneic skin grafts on day 0 and received PBS injections on days −1, 1, 4, and 6. The PJ19.1 monoclonal antibody treated mice (diamond symbols) received syngeneic (data not shown) and allogeneic skin grafts on day 0 and received injections of 500 μg of PJ19.1 monoclonal antibody i.p. on days −1, 1, 4, and 6. The percentage of mice with surviving skin grafts is plotted against days post transplantation of the skin grafts. There are five mice in the control group and four mice in the PJ19.1 antibody treated group.

FIG. 5 shows splenocyte cell counts from a control mouse (left panel) and a PJ19.1 monoclonal antibody treated mouse (right panel) as described in Example 8. The splenocytes were three color stained for CD4, CD8, and SPEX and analyzed by flow cytometry. Dots representing one count each of CD4+T cells are in the upper left quadrant of each panel, representations of CD8+T cells are shown in the lower right quadrant of each panel, and representations of B cells (CD4 negative and CD8 negative cells) are shown in the lower left quadrant of each panel. The numbers in each quadrant represent the percentage of each type of cells in the sample. Counts of the CD4+T cells, CD8+T cell, and B cell populations are not significantly increased or decreased in the PJ19.1 monoclonal antibody treated mouse compared to the control mouse.

### DETAILED DESCRIPTION OF THE INVENTION

The present inventor discovered, in part, that antibodies which immunoreact with an extracellular domain of a SPEX polypeptide are useful to inhibit transplant rejection and to decrease the expression of a SPEX polypeptide on lymphocytes. The present inventor further discovered, in part, a method of identifying antibodies that inhibit transplant rejection and that decrease the expression of SPEX on lymphocytes.

1. Definitions

As used herein, the term “syngeneic” refers to genetically identical or closely related organisms, cells, tissues, organs, and the like.

As used herein, a “syngeneic skin graft” refers to a skin graft wherein the host for the skin graft and the source of the skin graft are individuals that are genetically identical or sufficiently closely related such that the graft and the host do not interact antigeneically.

As used herein, the term “allogeneic” refers to organisms, cells, tissues, organs, and the like from, or derived from, individuals of the same species, but wherein the organisms, cells, tissues, organs, and the like are genetically different one from another.

As used herein, the term “xenograft” refers to a transplant in which the donor and recipient are of different species.

As used herein, an “allogeneic skin graft” refers to a skin graft wherein the host for the skin graft and the source of the skin graft are individuals of the same species that are sufficiently unlike genetically such that the graft and the host interact antigeneically. An allogeneic transplant is rejected in time in the absence of an intervention to inhibit transplant rejection.

As used herein, the term “transplant rejection” refers to a partial or complete destruction of a transplanted cell, tissue, organ, or the like on or in a recipient of said transplant.

As used herein, the term “host” refers to an organism (preferably the organism is a mammal), a tissue, organ, or the like that is the recipient of a transplanted cell, tissue, organ, or the like. The terms “host” and “recipient”, when referring to transplant hosts or recipients are used interchangeably herein.

As used herein, the term “globally depletes lymphocytes” refers to a decrease in the total counts of CD4+ T lymphocytes, CD8+ T lymphocytes, or B lymphocytes by 50% or more by administration of an immunosuppressive agent to a mammal compared to the corresponding lymphocyte counts in a control animal (which does not receive the immunosuppressive agent). The measurement of lymphocyte counts is typically carried out using blood, serum, or plasma samples. An immunosuppressive agent that does not “globally deplete lymphocytes” refers to an agent wherein administration of the agent to a mammal results in more than 50% of the total counts of CD4+ T lymphocytes, CD8+ T lymphocytes, and B lymphocytes being maintained (preferably 80% or more, more preferably 90% or more, and still more preferably 100% being maintained).

As used herein, an “amount therapeutically effective to inhibit transplant rejection”, in regard to an antibody that immunoreacts with an extracellular domain of a SPEX polypeptide, refers to an amount of the antibody that when administered to a transplant recipient, that amount inhibits, either partially or completely, rejection of the transplant over time. The antibody (and, optionally, a second transplant rejection inhibitor) can be administered before, during, and/or after transplantation.

As used herein, a “soluble heterologous polypeptide” is a polypeptide that is different from a SPEX polypeptide in terms of sequence (it is a non-SPEX polypeptide) and is more soluble in aqueous solution than is an extracellular domain of a SPEX polypeptide.

As used herein, the terms “isolated” and “purified” are used interchangeably and mean that the particular compound of interest is separated from other contaminating substances so as to be free, or essentially free, from impurities including toxic matter such as endotoxin. The meaning of “purified” herein discounts solutes, excipients, stabilizers, buffers, salts, acids, acid salts, pharmaceutically acceptable carriers, and the like which are often desirable in combination with an active ingredient (e.g., a monoclonal antibody that immunoreacts with an extracellular domain of a SPEX polypeptide) and are not considered an impurity.

2. SPEX

The discovery of SPEX is described in U.S. Ser. No. 10/831,622, filed Apr. 23, 2004 and U.S. Serial No. 06/467,206, filed Apr. 30, 2003; both applications are incorporated herein by reference. SPEX was described in a publication in Nature Immunology (2003, 4(7): 670-679), which is incorporated herein by reference. In the Nature Immunology paper, SPEX is referred to as B and T lymphocyte attenuator (BTLA).

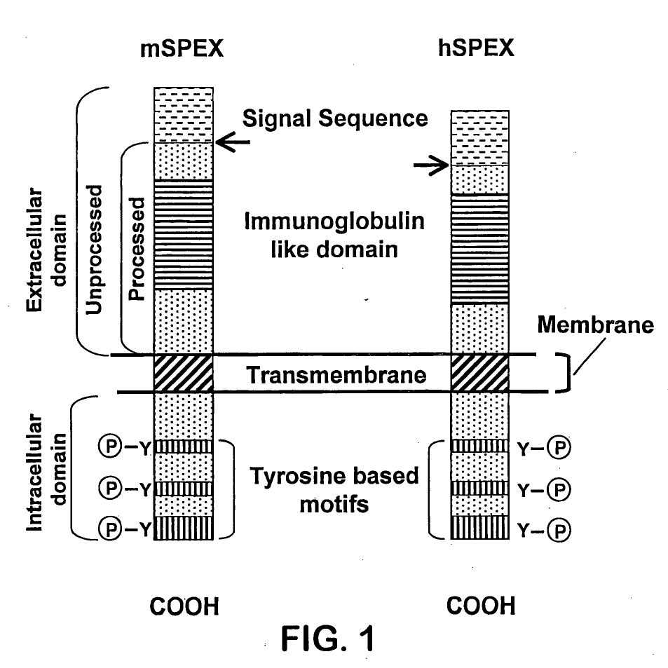

SPEX mRNA and polypeptide is expressed by lymphocytes including B and T lymphocytes, naïve B and T lymphocytes, thymocytes, activated B and T lymphocytes, splenic macrophages, antigen presenting cells (APCs) and mature bone marrow-derived dendritic cells (see, e.g., Han et al., (2004) Journal of Immunology 172: 5931-5939, incorporated herein by reference). The SPEX polypeptide is a multiple domain protein which is observed to be expressed at the cellular membrane. SPEX polypeptide includes an extracellular domain, a single transmembrane domain which is contemplated to anchor the polypeptide in the cellular membrane, and an intracellular domain. The SPEX polypeptide is a receptor for the B7x ligand (also known as B7-H4 and B7S1). The SPEX extracellular domain includes an immunoglobulin (Ig) like domain and the intracellular domain includes three tyrosine based signaling motifs. The tyrosine based signaling motifs are contemplated herein to be inhibitory signaling motifs which, for example, are contemplated to inhibit, or negatively modulate the activation of lymphocytes. SPEX is contemplated to be a type I receptor phosphoprotein of the immunoglobulin superfamily. A diagrammatic representation of mouse and human SPEX polypeptides is provided in FIG. 1.

Mice deficient in SPEX do not show apparent lymphocyte developmental defects; however, T cells from SPEX deficient mice are hyper responsive to anti-CD3 antibody stimulation of T cell activation. Based, in part, on this observation, SPEX is contemplated to be a negative regulator of lymphocyte activation, proliferation, and function.

3. Inhibition of Transplant Rejection

The present inventor made the surprising discovery that an antibody which immunoreacts with an extracellular domain of a SPEX polypeptide inhibits transplant rejection in a murine model of transplant rejection. Accordingly, one embodiment of the present invention provides a method of inhibiting transplant rejection in a mammal in need thereof, comprising: administering a transplant rejection inhibiting amount of an antibody to the mammal, wherein the antibody immunoreacts with an extracellular domain of a SPEX polypeptide.

A. SPEX Extracellular Domain

As discussed above, the present invention provides that an antibody which immunoreacts with an extracellular domain of a SPEX polypeptide, inhibits transplant rejection. Accordingly, the present invention provides SPEX polypeptides useful, for example, in the manufacture of antibodies that inhibit transplant rejection and SPEX polynucleotides useful, for example, in the manufacture of said SPEX polypeptides. Any SPEX polypeptide is useful as an immunogen for the manufacture of an anti-SPEX antibody. Preferred SPEX polypeptides comprise (or, alternatively, consist essentially of) at least six consecutive amino acids of the extracellular domain of a SPEX polypeptide. A highly preferred SPEX polypeptide comprises (or, alternatively, consists essentially of) an Ig like domain of the SPEX protein (see, e.g., SEQ ID NO:3 (human), 45 (murine), or 88(murine, allele b, see below)). The Ig like domain is highly preferred as the antigen for manufacturing anti-SPEX antibodies for use in the present invention.

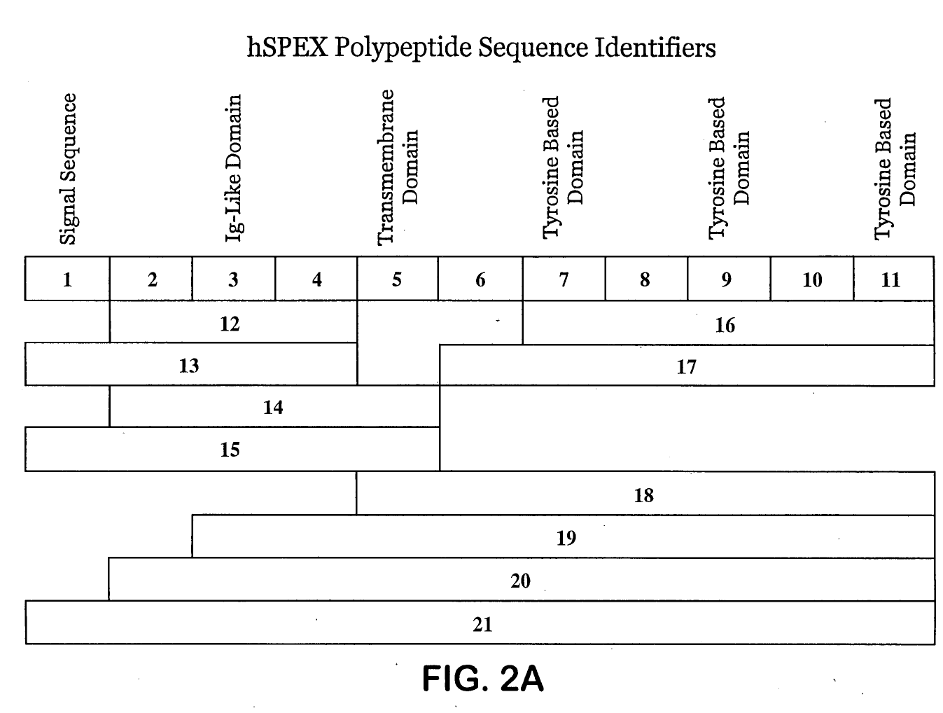

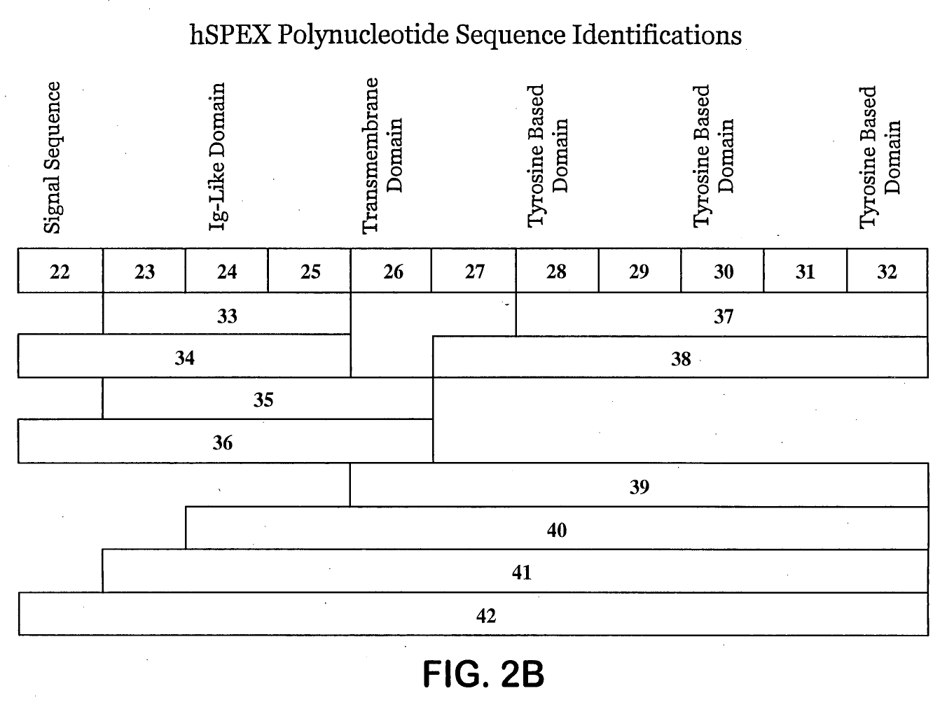

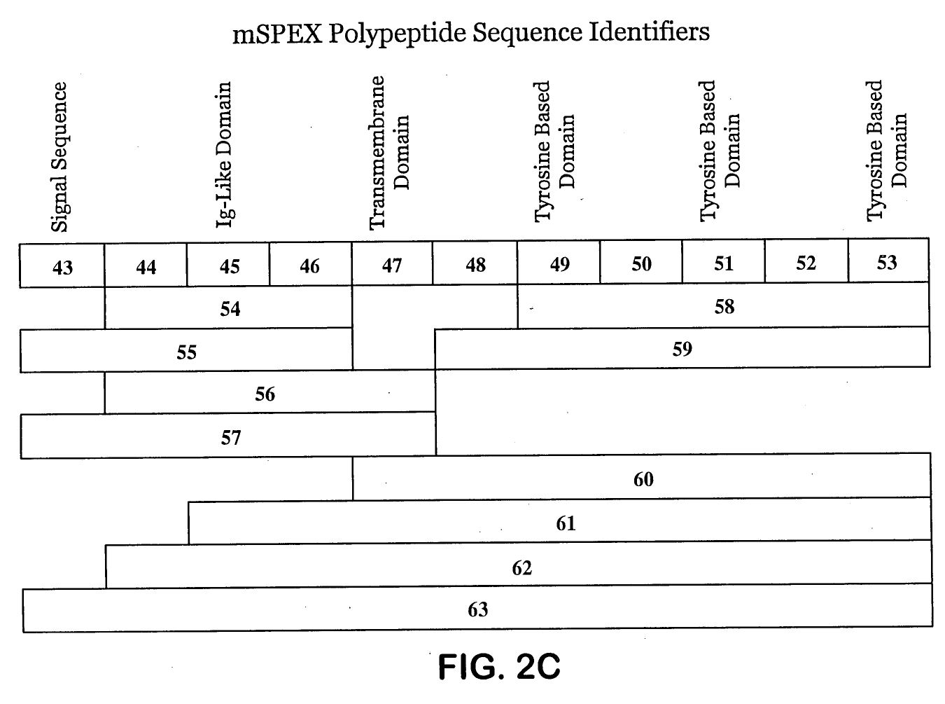

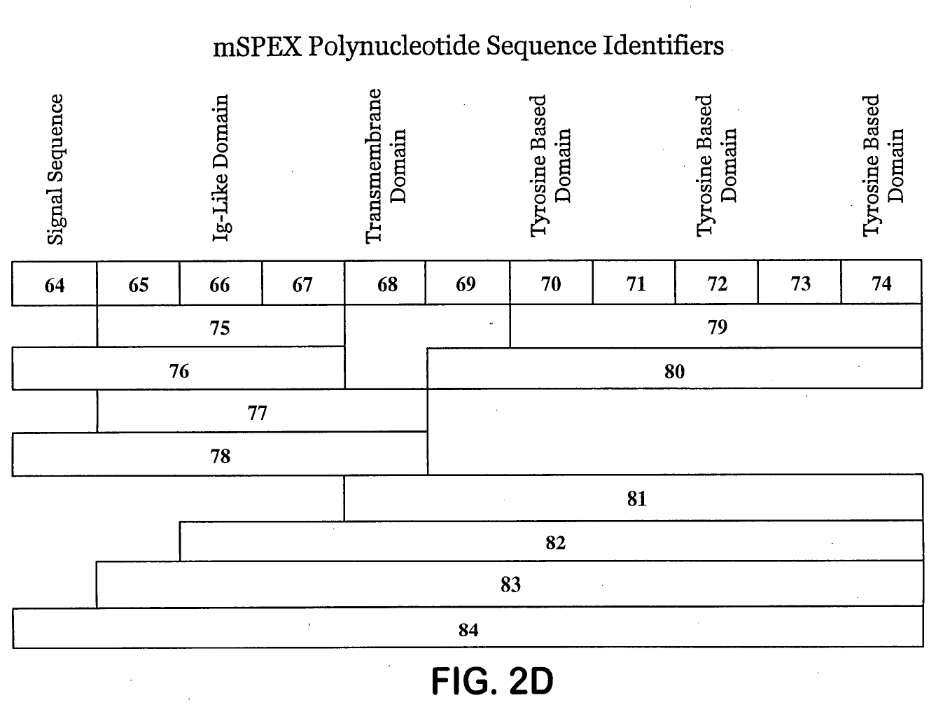

Certain embodiments of the present invention provide a SPEX polypeptide of mouse, human (preferred), or other mammalian origin. Examples of polypeptide, and corresponding polynucleotide sequences, with sequence identifiers, of human SPEX (hSPEX) and mouse SPEX (mSPEX) are set forth diagrammatically in FIGS. 2A-D. An allele of a mSPEX is also disclosed and is referred to herein as mSPEXb (only the extracellular portion of mSPEXb has been sequenced). An example of the unprocessed extracellular domain of mSPEXb is set forth in SEQ ID NO:85 (includes the signal sequence). An example of the processed extracellular domain of mSPEXb is set forth in SEQ ID NO:86 (without the signal sequence). The preferred Ig like domain of mSPEXb is set forth in SEQ ID NO:88.

As used herein, a highly preferred extracellular domain of a SPEX polypeptide comprises (alternatively, consists essentially of): SEQ ID NO:1, 2, 3, 4, 12, or 13 (from hSPEX). A preferred extracellular domain of a SPEX polypeptide comprises (alternatively, consists essentially of): SEQ ID NO:43, 44, 45, 46, 54 or 55 (from mSPEX) or SEQ ID NO:85, 86, or 86 (from mSPEXb). A still more highly preferred SPEX polypeptide comprises (alternatively, consists essentially of) the Ig like domain of human SPEX as set forth in SEQ ID NO:3.

In general, a polypeptide including six or more consecutive amino acid residues is capable of eliciting an immune response. Thus, certain embodiments provide an amino acid sequence comprising (or consisting essentially of) six consecutive amino acids of a SPEX extracellular domain (e.g., SEQ ID NO:13, 55, or 85). Typically, increasing the number of consecutive amino acid residues in the SPEX polypeptide enhances the immunogenic response to the peptide. Thus, certain embodiments provide, in increasing order of preference, a SPEX immunogen comprising 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or more consecutive amino acids of an extracellular domain of a SPEX polypeptide. Optionally, the SPEX immunogen comprises 6 to 9, 10 to 19, 20 to 29, 30 to 39, or 40 to 50 consecutive amino acids of the SPEX polypeptide.

One embodiment of the present invention provides a “SPEX fusion polypeptide” comprising an extracellular domain of a SPEX polypeptide operably linked with a soluble heterologous polypeptide. A preferred soluble heterologous polypeptide comprises a constant region of human IgG1, more preferably the human IgG1 hinge CH2, and CH3 domains. In one embodiment, a solubility promoting heterologous polypeptide comprises SEQ ID NO:97. Another embodiment provides a polypeptide comprising a SPEX polypeptide set forth in SEQ ID NO:3, SEQ ID NO:45, SEQ ID NO:88, SEQ ID NO:12, SEQ ID NO:54, or SEQ ID NO:86 operatively linked to a heterologous polypeptide including a constant region of human IgG1; preferably the human IgG1 hinge, CH2, and CH3 domains. Protein A binds the constant region of IgG1 with high affinity. Accordingly, a SPEX-human IgG1 fusion can be purified using protein A affinity chromatography.

I. Manufacture of a SPEX Polypeptide

In light of the present disclosure, a SPEX polypeptide of this invention can be made using a variety of techniques well known in the art for making a polypeptide. For example, a SPEX polypeptide can be manufactured by purification from natural sources (e.g., lymphocytes or spleen or thymus tissue). In another example, a SPEX polypeptide can be manufactured through expression of a polynucleotide encoding the SPEX polypeptide in a host cell including in bacterial (e.g., K12), eukaryotic, yeast, insect, plant, mammalian, Chinese hamster (e.g., CHO), murine, and human cells (e.g., using transfer of a recombinant SPEX expression system). See FIGS. 2B and 2D for examples of sequence identifiers of useful SPEX polynucleotides. In still another example, a SPEX polypeptide is manufactured using synthetic de novo methods. See FIGS. 2A and 2C (and SEQ ID NO:85-88) for examples of sequence identifiers of SPEX polypeptides (preferably an extracellular domain thereof) useful herein.

In preferred embodiments, the SPEX polypeptide is purified using methods known in the art for protein separation and purification. For example, in light of the present invention, one of ordinary skill in the art is able to use an anti-SPEX antibody disclosed herein to isolate or purify a SPEX polypeptide including specific fragments and domains thereof through affinity separation. Regarding the SPEX fusion polypeptide discussed above, protein A binds the constant region of IgG1 with high affinity. Accordingly, a SPEX-human IgG1 fusion can be purified using protein A affinity chromatography. The fusion polypeptide is preferably made using recombinant DNA techniques, polypeptide expression, and purification techniques that are well known in the art. A preferred “operable linkage” comprises a peptide bond. Alternatively, the soluble heterologous polypeptide can be operatively linked to the extracellular domain of the SPEX polypeptide using chemical crosslinking agents, compositions of which and use thereof are well known in the art.

Examples of useful amino acids residues (and nucleotide residues) for the manufacture of a SPEX polypeptide are provided in the World Intellectual Property Organization (WIPO) Handbook on Industrial Property Information and Documentation, Standard ST.25: Standard for the Presentation of Nucleotide and Amino Acid Sequence Listings in Patent Applications (1998), including Tables 1 through 6 in Appendix 2; hereinafter referred to as “WIPO Standard ST.25 of 1998”, incorporated herein by reference.

B. Antibodies that Immunoreact with an Extracellular Domain of a SPEX Polypeptide

One embodiment of the present invention provides an antibody that immunoreacts with an extracellular domain of a SPEX polypeptide. The present antibodies are useful, for example, to decrease lymphocyte SPEX expression and to inhibit transplant rejection. The term “antibody” is meant to include any form of antibody, including intact antibodies molecules and/or an immunologically active portion or fragment of an antibody molecule. Antibodies and active fragments thereof are well known in the art, for example: IgG, IgM, IgE, polyclonal, monoclonal, Fab, Fab′, F(ab′)₂, F(v), single chain antibody (SCA), single chain Fab, humanized, hybrid, and the like antibodies). It is preferred that the antibody is isolated. It is also preferred that the antibody comprises a recombinant, chimeric, or otherwise non-naturally occurring antibody. In certain embodiments it is preferred that the antibody is multivalent (comprises two or more epitope binding sites), for example, a bivalent antibody. A preferred antibody is an anti-SPEX monoclonal antibody, preferably a human or humanized anti-SPEX monoclonal antibody, which immunoreacts with an extracellular domain of the SPEX polypeptide.

I. Antibody Manufacture

Methods of manufacturing an antibody given a specific antigen are well known in the art. The present invention provides SPEX antigens (i.e., immunogen) useful for manufacture of anti-SPEX antibodies that immunoreact with an extracellular domain of a SPEX polypeptide. Thus, in light of the present disclosure, one of ordinary skill in the art is able to manufacture an anti-SPEX antibody useful in the present invention. Antibodies are commonly manufactured, for example: in animals (e.g., rabbit, mouse, hamster, sheep, goat, horse, bovine); in cells, primarily cell culture, (e.g., bacteria, plant, algae, insect, mammalian, murine, hybridoma, and human cells); by phage display; and by epitope cloning into antibody scaffold vectors and gene transfer into any of a variety of cell types (e.g., bacteria, plant, algae, insect, mammalian, murine, and human).

4. Pharmaceutical Compositions

One embodiment of the present invention provides a “SPEX pharmaceutical composition” comprising an antibody that immunoreacts with an extracellular domain of a SPEX polypeptide, wherein the antibody is capable of inhibiting transplant rejection, and wherein the antibody is in an amount therapeutically effective to inhibit transplant rejection. A preferred antibody that immunoreacts with the extracellular domain of a SPEX polypeptide and is capable of inhibiting transplant rejection is PJ19.1 which immunoreacts with the extracellular domain of mSPEX polypeptide (e.g., SEQ ID NO:55).

Preferred SPEX pharmaceutical compositions are prepared to minimize contaminants. Preferred SPEX pharmaceutical compositions are also prepared to minimize allergic and toxic reactions in a recipient to which the compositions are administered. In one embodiment, it is preferred that the SPEX pharmaceutical composition is pyrogen (e.g., endotoxin) free, or essentially pyrogen free.

In preferred embodiments, a SPEX pharmaceutical composition further comprises a second transplant rejection inhibitor (meaning one or more transplant rejection inhibitor(s) in addition to the anti-SPEX antibody). Preferred second transplant rejection inhibitors include, but are not limited to: CsA, rapamycin, FK506, corticosteriods, and antibodies that immunoreact with the IL-2 receptor.

A. Preparation of a Pharmaceutical Composition

In light of the present invention, a SPEX pharmaceutical composition (comprising an antibody that immunoreacts with an extracellular domain of a SPEX polypeptide) may be manufactured in any manner that is known in the art (including, e.g., by conventional mixing, dissolving, granulating, dragee-making, emulsifying, encapsulating, entrapping, lyophilizing, or suspending processes). It is preferred that manufacture is according to Good Manufacturing Practice, the procedures and regulations of which are known in the art.

In certain embodiments, the SPEX pharmaceutical composition is manufactured to further include a pharmaceutically acceptable carrier, excipient, auxiliary, preservative, or other ingredient (referred to collectively herein as a “pharmaceutically acceptable carrier”). The term “carrier” refers herein to a “pharmaceutically acceptable carrier”. Preferably, a pharmaceutically acceptable carrier is suitable for administration to a human or a non-human mammal. Further details on techniques for formulation and administration of pharmaceutical compositions may be found in the latest edition of Remington's Pharmaceutical Sciences (Maack Publishing Co., Easton, Pa.).

Fluid carriers may include aqueous solutions, preferably in physiologically compatible buffers (e.g., Hanks' solution, Ringer's solution, or physiologically buffered saline). Fluid carriers also include non-aqueous and oily suspensions. Suitable lipophilic solvents or vehicles may include fatty oils (e.g., sesame oil, synthetic fatty acid esters, ethyl oleate, triglycerides, or liposomes). Useful liposomes include cationic liposomes, anionic liposomes, and liposomes with neutral charge density. Viscosity enhancing agents may be included (e.g., sodium carboxymethyl cellulose, sorbitol, or dextran). Stabilizers, adhesives, or agents which increase solubility may also be included. Additional inert ingredients may include any or all of gum arabic, syrup, lanolin, or starch. Another excipient which may be used is polyethylene glycol (PEG). PEG can be admixed with the formulation or linked to the anti-SPEX antibody molecule itself. PEG may be useful, for example, as a dehydrating or concentrating agent. Accordingly, carriers may be aqueous, non-aqueous (hydrophobic), or amphiphilic. Delayed release and/or sustained release carriers, and the pharmaceutical formulations thereof, are known in the art and can be used in embodiments herein, in light of the present disclosure.

The SPEX pharmaceutical composition may be provided as a salt and can be formed with an acid (e.g., hydrochloric, sulfuric, acetic, lactic, tartaric, malic, succinic, and the like). In other embodiments, the SPEX pharmaceutical composition may be a lyophilized powder which is preferably combined with buffer prior to use.

B. Administering a Pharmaceutical Composition

In certain embodiments of the present invention, a SPEX pharmaceutical composition (comprising an antibody that immunoreacts with a SPEX extracellular domain) is administered to a patient in need of treatment for transplant rejection. The pharmaceutical compositions encompassed by this invention may be administered by any desirable route including, but not limited to, oral, intravenous, intramuscular, nasal, intratracheal, intra-articular, intra-arterial, intramedullary, intrathecal, intraventricular, transdermal, subcutaneous, intraperitoneal, intranasal, intratumoral, enteral., topical, sublingual, vaginal, or rectal routes of administration. In light of the present invention, one of ordinary skill in the art is able to select a suitable route for administering a SPEX pharmaceutical composition to a patient.

Factors considered for the route of administration may include the location of the transplant and the condition of the patient. Also, in light of the present invention, one of ordinary skill in the art is able to select suitable SPEX pharmaceutical compositions which are desirable for a particular route of administration including formulations with carriers, excipients, auxiliaries, inert ingredients, and the like.

Methods of administering include, but are not limited to injection, infusion, administration by catheter, microinjection, particle mediated (biolistic) transfer, intubation, inhalation, ingestion, diffusion from a matrix, and the like. In one example, the SPEX pharmaceutical composition may be administered by injecting the composition into the afferent blood supply of a transplanted organ or tissue. A preferred method of administering a SPEX pharmaceutical composition comprises intraperitoneal injection.

C. Dosage of a Pharmaceutical Composition

Pharmaceutical compositions suitable for use in the invention include an antibody that immunoreacts with an extracellular domain of a SPEX polypeptide in an effective amount, or dose, to achieve an embodied purpose (e.g., in treating a patient having or in need of a transplant). In light of the present invention and knowledge in the art, the determination of an effective dose is well within the capability of those skilled in the art.

A therapeutically effective dose or range can be estimated initially either in cell culture assays or in animal models; usually in mice, rats, rabbits, dogs, pigs, or non-human primates. The animal model may also be used to determine the preferred concentration range and route of administration. Such information can then be used to select preferred doses and routes for administration in humans. A preferred animal model comprising murine skin grafting is discussed below.

Therapeutic efficacy and toxicity may be determined by standard pharmaceutical procedures in cell cultures, experimental animals, or other transplant model systems. For example, the ED50 (the dose therapeutically effective in 50% of the population) and the LD50 (the dose lethal to 50% of the population) can be determined in a model system. The dose ratio between toxic and therapeutic effects is the therapeutic index, which may be expressed as the ratio, LD50/ED50. SPEX pharmaceutical compositions which exhibit large therapeutic indices are preferred. The SPEX pharmaceutical composition optionally includes the use of a second transplant rejection inhibitor to enhance or increase the therapeutic index. The data obtained from the model system(s) is used in formulating a range of dosage for human use. The dosage contained in such compositions is preferably within a range of circulating concentrations that include the ED50 with low toxicity or, more preferably, essentially no toxicity. In one embodiment, the SPEX pharmaceutical composition includes a dose of an antibody that immunoreacts with an extracellular domain of a SPEX polypeptide that includes a therapeutically effective ED50. It is more preferred that the present SPEX pharmaceutical composition includes a toxicity or LD50 that is acceptable for administering the SPEX pharmaceutical composition to a patient (a human or, optionally, a non-human mammal) after taking into account the relative condition of the patient and the need that the patient has for treatment with a transplant rejection inhibitor. Accordingly, the dosage of the SPEX pharmaceutical composition that is used in a patient is preferably determined by the practitioner, in light of factors related to the patient that requires treatment.

Dosage and administration are adjusted to provide sufficient levels of the active moiety(ies) (e.g., circulating and/or local concentration) or to maintain the desired effect. Factors which may be taken into account include the severity of the disease state, general health of the subject, age, weight, gender, diet, time and frequency of administration, drug combination(s), reaction sensitivities, tolerance to therapy, and response to therapy. In general, SPEX pharmaceutical compositions may be administered, for example; once a day, every other day, every 3 to 4 days, every week, once every two weeks, once a month, once every several months, or yearly.

Dosage amounts include, for example, from 0.1 mg/kg to 20 mg/kg per dose, preferably from 2.5 mg/kg to 20 mg/kg per dose, of an antibody that immunoreacts with an extracellular domain of a SPEX polypeptide.

5. Decreasing Lymphocyte Expression of SPEX Polypeptide

One embodiment of the present invention provides a method of decreasing an expression of a SPEX polypeptide on a lymphocyte which expresses the SPEX polypeptide, comprising contacting the lymphocyte with an antibody that immunoreacts with an extracellular domain of the SPEX polypeptide, thereby leading to a down regulation of the SPEX polypeptide on the surface of the lymphocyte.

Preferred lymphocytes include, but are not limited to CD4+ T cells, CD8+ T cells, and B cells. A preferred antibody is a monoclonal antibody. It is also preferred that the present antibody is a human or a humanized antibody. The present antibody immunoreacts with the extracellular domain of the SPEX polypeptide (the antibody is particularly preferred to immunoreact with the Ig like domain), and, optionally, the antibody does not immunoreact with an intracellular domain and/or an intracellular domain of the SPEX polypeptide. In certain embodiments the present antibody immunoreacts with SEQ ID NOs:3, 12, 13, 45, 54, 55, 85, 86, or 88 and, optionally, does not immunoreact with SEQ ID NOs:17, 18, 59, or 60.

6. Identifying Antibodies that Inhibit Transplant Rejection

One embodiment of the present invention provides a method of identifying an antibody that inhibits transplant rejection in a mammal, comprising: providing an amino acid sequence comprising an epitope of an extracellular domain of a SPEX polypeptide, wherein the epitope is at least six amino acids in length; producing antibodies to the amino acid sequence; and screening the antibodies in a model of transplant rejection to identify antibodies that inhibit transplant rejection.

A. Mammalian Model System of Transplant Rejection

Techniques are well known to one of ordinary skill in the art for the transplantation of numerous cell, tissue, and organ types including, but not limited to: pancreatic islet transplantation, corneal transplantation, bone marrow transplantation, stem cell transplantation, skin graft transplantation, skeletal muscle transplantation, aortic and aortic valve transplantation, and vascularized organ transplantation including, but not limited to: heart, lung, heart and lung, kidney, liver, pancreas, and small bowel transplantation (see, e.g., Experimental Transplantation Models in Small Animals (1995) Publisher T&F STM, 494 pages). The present invention is not limited by the particular variety of transplantation.

In general, transplantation between a non-syngeneic donor and recipient, in the absence of a transplant rejection inhibitor results in transplant rejection characterized by the partial or complete, typically progressive, destruction of the transplanted cells, tissue, or organ(s). Accordingly, any non-syngeneic (e.g., allogeneic or xenogeneic) transplantation is useful herein as a model system of transplant rejection. A preferred model system of transplant rejection inhibition comprises a murine allogeneic skin graft.

In one embodiment, non-syngeneic transplants are performed on two groups of mammals, wherein a first group is not treated (the control group) with a transplant rejection inhibitor and a second group is treated with a SPEX pharmaceutical composition comprising an antibody that immunoreacts with an extracellular domain of a SPEX polypeptide. The progress of the transplants are monitored over time for the percentage of transplant rejection (destruction of the transplant). The anti-SPEX antibody is capable of inhibiting transplant rejection, for example, if the percentage of transplant rejection is reduced or eliminated for a given time period or if the transplant survives for a longer period of time for a given amount of transplant rejection in the treated versus group the non-treated controls.

In another embodiment, an antibody that immunoreacts with an extracellular domain of the SPEX polypeptide and a second transplantation rejection inhibitor is administered to a first group of mammals and the second transplantation rejection inhibitor is administered to a second group of mammals (the second inhibitor is not an anti-SPEX antibody). The present embodiment allows, for example, for the detection of a synergistic effect of the anti-SPEX antibody and the second inhibitor in the inhibition of transplant rejection.

The following examples are intended to illustrate, but not limit, the invention.

### EXAMPLES

1. Identification and Cloning of mSPEX

Positive selection is a developmental process in which immature thymocytes receive maturation signals as a consequence of T cell antigen receptor (TCR) recognition of MHC/self-peptide complexes expressed by thymic stroma. Exposure of immature thymocytes to low concentrations of the pharmacologic activators phorbol ester (e.g., PMA) and/or ionomycin induces the survival and differentiation of double positive (DP) thymocytes in vitro and provides an accepted model system of positive selection of thymocytes in vivo.

Using nucleic acid microarrays, the inventors identified changes in nucleic acid expression during positive selection of thymocytes relative to unstimulated thymocytes. TCRα-chain deficient thymocytes (murine) were cultured in medium in the presence or absence of 0.2 ng/ml PMA and 0.2 mg/ml ionomycin providing activated and non-activated thymocytes respectively. TCRα-chain deficient thymocytes are blocked at the DP stage of development due to a genetic mutation. Gene expression in these cells, after stimulation, is characteristic of developing thymocytes. After incubating the cells for 6 hours, the poly-A+ RNA was isolated using RNeasy RNA and Oligotex mRNA kits (Qiagen). The poly-A+ RNA was subjected to comparative cDNA array analysis using the Mouse 1.02 Array per manufacturer's instructions (Incyte Genomics). Signal analysis of the microarrays was performed using GEMTools analysis software (Incyte Genomics).

The inventors selected two sequences for further study based upon the ratio of expression of the sequences in the stimulated thymocytes over the unstimulated thymocytes. The ratio of expression was determined from the normalized signals of hybridization of each labeled cDNA to the ESTs AA184189 and AA177302, respectively, which ESTs were included on the Mouse Array. The expression of each sequence in the stimulated thymocytes was increased 9.1 fold and 6.6 fold for sequences hybridizing to EST AA184189 and EST AA177302, respectively. These ESTs on the Mouse 1.02 Array originated from a murine library called the Soares mouse 3NbMS library which in turn was derived from spleens of 4 week old C57BL/6J mice.

The inventors determined that the ESTs AA184189 and AA177302 are linked using BLAST software (NCBI) to compare the sequences of the ESTs with a database of murine nucleic acid sequences (each EST corresponds to a common sequence in the murine sequence database). Thus, ESTs AA184189 and AA177302 are determined to be part of a more complete nucleic acid. Using the 5′-most EST (AA177302), the inventors cloned a full-length gene by rapid amplification of cDNA ends (SMART RACE cDNA amplification kit, Clontech) using cDNA prepared from stimulated thymocytes as a template in the reaction.

The present gene is designated herein as the mouse spleen expressed gene (mSPEX gene). An exemplary mSPEX nucleic acid is set forth in SEQ ID NO:105 and includes a coding region set forth in SEQ ID NO:84. A BLAST search of GenBank reveals that the SPEX cDNA encodes a novel protein. An exemplary mSPEX polypeptide encoded by an mSPEX nucleic acid is set forth in SEQ ID NO:63. A signal sequence (e.g., SEQ ID NO:43) is typically cleaved during cellular processing to form an exemplary mature mSPEX polypeptide set forth in SEQ ID NO:62. Using sequence comparison between the mSPEX polypeptide and the Pfam family of proteins database (NCBI), the inventors identified an immunoglobulin like domain in a predicted extracellular portion of the polypeptide; however, the inventors observed that SPEX lacks close homology to any particular immunoglobulin containing superfamily member.

2. Identification and Cloning of a hSPEX cDNA

Using sequence comparisons of the mSPEX sequence to human sequences in the GenBank database the inventors determine that the human ESTs AI792952 and AA931122 align with the mSPEX polynucleotide and that the human ESTs correspond to a single human clone in the GenBank database. A bacterial stab (AI792952) containing the human clone, as well as numerous contaminating clones, was purchased from the IMAGE Consortium (Lawrence Livermore National Laboratory, Livermore, Calif.). A human gene sequence homologous to mSPEX is purified from the stab, subcloned, and sequenced. An exemplary hSPEX nucleic acid sequence is set forth in SEQ ID NO:104 and includes a coding region set forth in SEQ ID NO:42. An exemplary hSPEX polypeptide encoded by an hSPEX nucleic acid sequence is set forth in SEQ ID NO:21. A signal sequence (e.g., SEQ ID NO:1) is typically cleaved during cellular processing of hSPEX protein to form an exemplary mature hSPEX polypeptide set forth in SEQ ID NO:20.

3. Manufacture of Rat Anti-mSPEX Monoclonal Antibodies

An isolated expression vector, referred to herein as p-mSPEX-Ig, was manufactured comprising a coding region encoding a mSPEX extracellular domain and the hinge, CH2, and CH3 domains of human IgG1. The p-mSPEX-Ig vector is transfected into 293 cells in culture. The p-mSPEX-Ig transfected 293 cells express and secrete the mSPEX-Ig fusion protein (SEQ ID NO:99) as assessed by a Western blot probed with ani-humanIgG1 antibody. The mSPEX-Ig fusion protein is purified by protein A affinity chromatography from supernatants of transfected 293 cells.

Rats are immunized in the base of the tail with the purified recombinant mSPEX-Ig emulsified in CFA to produce monoclonal antibodies. Procedures and techniques for the production of antibodies, including monoclonal antibodies, to a given antigen are well known in the art. The medial iliac lymph nodes are harvested two weeks later and fused with YB2/0 cells by standard methods. Antibodies thus produced are screened for immunoreactivity with mSPEX by FACS using DPK and 293 cells that are transfected to express a cell surface mSPEX-YFP fusion protein (YFP is an abbreviation for yellow fluorescent protein). The DPK and 293 cells used for the screening procedure are transfected with an isolated expression vector construct made from cloning a mSPEX gene insert into the pEYEP-N1 vector (Clontech). The pEYEP-N1 vector supplies an YFP tag and expression of a mSPEX insert from pEYEP-N1 results in the expression of a fusion protein including a mSPEX polypeptide operably linked with a YFP tag.

Screening continues until one or more monoclonal antibody that specifically immunoreacts with the extracellular domain of mSPEX is identified in the screening process. The YFP tag allows verification that transfected cells express the mSPEX-YFP fusion protein at the cell surface and allows the correlation of the relative level of cellular antibody binding to mSPEX-YFP fusion protein expression. Three hybridomas: PK3, PK18, and PK23 are obtained from a first screening process. The hybridomas are optionally re-cloned and antibody is purified using standard methods.

4. Manufacture of Rat Anti-hSPEX Monoclonal Antibodies

A monoclonal antibody that immunoreacts with the extracellular domain of a hSPEX polypeptide is prepared using the methods disclosed in Example 3, except that 1) a p-hSPEX-Ig expression vector comprising a hSPEX extracellular domain (including the SPEX signal sequence) and the hinge, CH2, and CH3 domains of human IgG1 is prepared and used to produce monoclonal antibodies in rats and 2) a hSPEX polynucleotide encoding the extracellular domain of a hSPEX polypeptide is cloned into a pEYEP-N1 vector and expressed in DPK and 293 cells to screen for monoclonal antibodies using the FACS-YFP procedure disclosed above. The monoclonal antibodies produced specifically immunoreact with the extracellular domain of hSPEX.

5. Manufacture of Mouse Anti-mSPEX Monoclonal Antibodies

Because there are multiple alleles known for murine SPEX (mSPEX and mSPEXb) it is possible to raise mouse anti-mouse SPEX antibodies in a mouse host without encountering self-tolerance. To raise a mouse monoclonal antibody against mSPEX, the procedures for Example 3 were followed except that the murine SPEX gene for the p-mSPEX-Ig fusion is derived from C57BL6 mice (which have the mSPEX allele) and immunizations are performed in BALB/c mice (which have the mSPEXb allele). Many anti-mSPEX antibodies with immunoreact with the extracellular domain of mSPEX were produced in a first screening process including, but not limited to: PJ19.1 and PJ196.

6. Manufacture of Mouse Anti-hSPEX Monoclonal Antibodies

To raise monoclonal antibodies that immunoreact with an extracellular domain of hSPEX, the procedures of Example 4 are followed except that mice, instead of rats, are immunized with a hSPEX-Ig fusion polypeptide (SEQ ID NO:98). The monoclonal antibodies produced specifically immunoreact with the extracellular domain of hSPEX.

7. Inhibiting Graft Rejection

It is determined that administering an antibody that immunoreacts with the extracellular domain of a SPEX polypeptide inhibits graft rejection. B6 mice (having the mSPEX allele, but not the mSPEXb allele) are injected intraperitoneally with 500 μg of the monoclonal antibody PJ19.1 (which immunoreacts with the extracellular domain of mSPEX, but not mSPEXb) on days −1, 1, 4, and 6 (relative to receiving a skin graft). Age and sex-matched control mice groups received injections of physiologically buffered saline (PBS) or (in parallel experiments) mouse IgG. Individual mice received syngeneic B6 and allogeneic BALB/c skin grafts on day 0. This particular strain combination of skin grafts is highly resistant to the induction of transplantation tolerance; thus, the induction of transplantation tolerance (i.e., the inhibition of transplantation rejection) by a transplantation inhibitor in this system is highly significant. Of note, the PJ19.1 antibody immunoreacts with the extracellular domain of mSPEX in the B6 transplant recipients, but not the mSPEXb in the allogeneic skin grafts from the BALB/c donors.

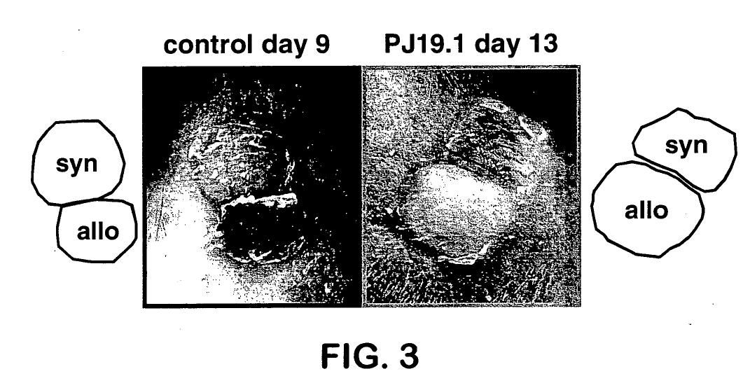

Graft rejection as a percentage of the total graft (percentage of graft surface area) was monitored from day 7 through day 16 with photographs taken daily. The day of graft rejection was determined as the day of 90% or more of tissue destruction. As shown in FIG. 3, no rejection of the syngeneic skin grafts was observed during the course of the experiment (sixteen days total). Also as shown in FIG. 3, the allograft (allogeneic skin graft) was rejected in a control mouse (PBS injection) shown at day nine, but rejection of the allograft was inhibited in a mouse treated with the PJ19.1 monoclonal antibody as described above and shown at day 13.

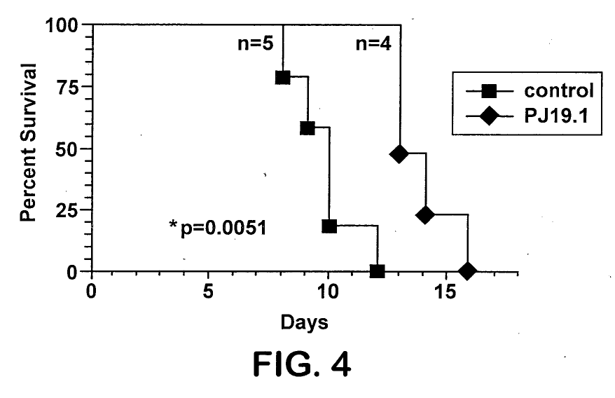

FIG. 4 provides a plot of the percentage of mice in experimental and control groups (see below) with surviving grafts versus the number of days for two groups of mice. A first group of five mice were administered PBS (the control group) at days −1, 1, 4, and 6 relative to each mouse receiving a syngeneic and an allogeneic skin graft, as described above. A second group of four mice were each administered 500 μg of the monoclonal antibody PJ19.1 (the experimental group) at days −1, 1, 4, and 6 relative to each mouse receiving a syngeneic and an allogeneic skin graft. Photographs of the skin grafts were taken daily until the last graft was rejected. The number of animals with surviving allogeneic skin graphs was recorded on a percentage basis and plotted against days post skin graft. As shown in FIG. 4, the median time for rejection (90% or more destruction) of the allogeneic grafts in the control PBS treated mice was day 10 (median), while the median time of rejection of the allogeneic grafts was inhibited in the PJ19.1 treated mice until day 13.5 (median) which is seven days after the last injection of antibody). Survival fractions were determined using the Kaplan-Meier method. Comparison of survival curves was performed using the logrank test provided by PRISM4 software (statistical software). Median survival is also calculated using the PRISM4 software. The difference between the control and the PJ19.1 treated mice in terms of median time to rejection is highly significant with a p value of 0.0051. It is contemplated that continuing the treatment with the PJ19.1 monoclonal antibody beyond day 6 post transplantation (e.g., every day or every other day administration) could result in a continued inhibition of allograft rejection.

8. Anti-SPEX Antibody does not Globally Deplete Lymphocytes

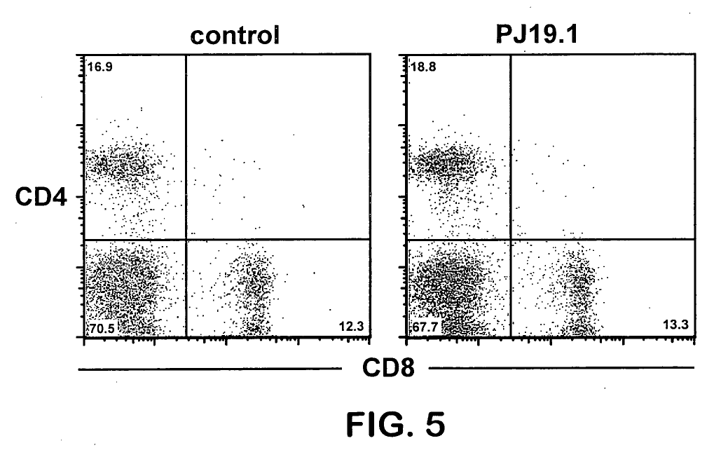

Example 7 is repeated except that the PBS and PJ19.1 treated mice are sacrificed 15 days after skin grafting (9 days after the last injection of antibody) and the spleen cells are analyzed for total B, CD4+T and CD8+T cell counts in each group of animals. As shown in FIG. 5, the frequency and number (data not shown) of CD4+T, CD8+T, and B cells is similar in the control (PBS) and PJ19.1 antibody treated mice. Accordingly, administration of an antibody that immunoreacts with the extracellular domain of a SPEX polypeptide does not globally deplete lymphocyte populations of CD4+T, CD8+T, or B cells.

9. Anti-SPEX Antibody Down-Regulates Lymphocyte SPEX Expression

Samples of B, CD4+T, and CD8+T cells from Example 8 (the samples are collected separately from control mice and from PJ19.1 treated mice) above are stained in vitro with PJ196 (which immunoreacts with the extracellular domain of SPEX) and secondary antibody (with immunoreacts with both PJ19.1 and PJ196 monoclonal antibodies). It is observed, data not shown: 1) that there is no global depletion of B, CD4+T, and CD8+T cells in the PJ19.1 treated mice, 2) that B, CD4+T, and CD8+T cells are coated in vivo with the PJ19.1 antibody in mice that were treated with PJ19.1, and 3) that the total cell surface expression of SPEX is decreased on the B, CD4+ T, and CD8+T cells in the PJ19.1 treated mice as compared to the PBS or IgG treated mice. Accordingly, administering a monoclonal antibody that immunoreacts with the extracellular domain of a SPEX polypeptide decreases the expression of SPEX on the surface of lymphocytes including B, CD4+ T, and CD8+ T cells.

10. Identification of Amino Acid Residues in the Extracellular Domain of SPEX Important to Antibody Immunoreactivity

Example 10 discloses certain, but not necessarily all, of the amino acid residues in the extracellular domain of mSPEX that are important to the immunoreactivity of the PK18, PK3, PJ196 and PJ19.1 antibodies.

The PK18, PK3, PJ196, and PJ19.1 antibodies are each raised against the mSPEX allele of murine SPEX and immunoreact with the extracellular domain of the mSPEX polypeptide, but do not immunoreact with mSPEXb polypeptide. There are differences in the amino acid sequence of the extracellular domain of the mSPEX allele compared to the mSPEXb allele at eleven amino acid residue positions as indicated in Table 1 below. Mutated mSPEX polypeptides are produced by independently mutating a polynucleotide encoding the mSPEX polypeptide such that a mSPEX polypeptide is produced with substitutions from the mSPEXb sequence at each residue in which there was a difference between the mSPEX and mSPEXb polypeptides (see Table 1 below). The mutations were made independently with the exception of amino acid residue positions 45 and 47 which were both mutated together forming a T45N/T47K double mutant.

TABLE 1
SEQUENCE DIFFERENCES BETWEEN THE EXTERNAL
DOMAINS OF MSPEX AND MSPEXB POLYPEPTIDES

| | | | MSPEX Mutant |
|---|---|---|---|
| Position | mSPEX Allele | mSPEXb Allele | Polypeptide |
| 41 | Proline (P) | Glutamate (E) | P41E |
| 45/47 | Threonine (T)/ | Asparagine (N)/ | T45N/T47K |
| | Threonine (T) | Lysine (K) | |
| 52 | Glutamine (Q) | Histidine (H) | Q52H |
| 55 | Arginine (R) | Tryptophan (W) | R55W |
| 63 | Glutamine (Q) | Glutamate (E) | Q63E |
| 74 | Asparagine (N) | Histidine (H) | N74H |
| 85 | Cysteine (C) | Tryptophan (W) | C85W |
| 91 | Serine (S) | Glycine (G) | S91G |
| 102 | Glutamine (Q) | Arginine (R) | Q102R |
| 143 | Threonine (T) | Arginine (R) | T143R |

Residue positions 52, 55, 63, 74, 85, 91, and 102 are included in the Ig like domain of the mSPEX polypeptide.

The mutated mSPEX polypeptides are analyzed for changes in the binding of each of the PK18, PK3, PJ196, and PJ19.1 antibodies to demonstrate which of the amino acids that differed between the extracellular domains of mSPEX and mSPEXb polypeptides, respectively, are important to the immunoreactivity of each antibody with the mSPEX polypeptide.

Jurkat T cells (a cell line of human origin) are transfected by electroporation with polynucleotide constructs encoding each mSPEX mutant polypeptide (see Table 1 above) fused at the carboxy-terminus of the mSPEX mutant with a yellow fluorescent protein (YFP). Controls transfections are performed with wild-type mSPEX-YFP fusion polynucleotides as well. Transfected cells comprising each construct (each mutant mSPEX-YFP or wild-type mSPEX-YFP) are divided into aliquots and then reacted with anti-SPEX antibodies (reacted in separate aliquots with PK18, PK3, PJ196, or PJ19.1) and then all of the samples are reacted with secondary antibodies. The secondary antibodies are linked to PE fluorochrome (FL2, herein) for detection by flow cytometry analysis.

Thus, aliquots of cells are produced wherein each aliquot expresses wild-type mSPEX or one of the mutant mSPEX polypeptides listed in Table 1 above on the cell surface of the Jurkat T cells and aliquots of each of these are reacted separately with PK18, PK3, PJ196, or PJ19.1. FACS analysis is performed by electronically gating the cells on a narrow range of YFP⁺ cells (fluorochrome 1 positive, or FL1⁺) and then measuring the amount of FL2 fluorescence. A decrease in the amount of FL2 fluorescence for a given primary antibody (PK18, PK3, PJ196, or PJ19.1) immunoreacting with each given mSPEX polypeptide expressing Jurkat T cell indicates that wild-type residue in the mSPEX polypeptide is part of the epitope that specifically immunoreacts with the antibody. The results of the above described experiment are tabulated in Table 2, below.

TABLE 2
RESIDUES OF MSPEX IMPORTANT TO
ANTIBODY IMMUNOREACTIVITY

| Position | PK3 | PK18 | PJ19.1 | PJ196 |
|---|---|---|---|---|
| 41 | − | +++ | − | ++ |
| 45/47 | + | ++ | − | ++ |
| 52 | − | − | − | − |
| 55 | ++ | − | − | − |
| 63 | − | − | − | − |
| 74 | − | − | − | − |
| 85 | − | ++ | +++ | − |
| 91 | − | − | ++ | − |
| 102 | − | − | − | − |
| 143 | − | − | − | − |

In Table 2, a minus (−) sign indicates that mutation of the respective amino acid position in the extracellular domain of mSPEX does not decrease binding of the antibody for the mutant mSPEX, a plus (+) sign indicates an arbitrary scale (from (+) being the lowest to (+++) being the greatest) of the extent of decrease in binding of each antibody for a mSPEX mutated at each respective amino acid position in the extracellular domain of the mSPEX polypeptide when compared to wilt-type SPEX.

Based upon the data shown in Table 2 above, it is observed that the PK3 immunoaffinity epitope includes residues 45/47, and 55; the PK18 immunoaffinity epitope includes residues 41, 45/47, and 85; the PJ19.1 immunoaffinity epitope includes residues 85 and 91; and the PJ196 immunoaffinity epitope includes residues 41 and 45/47. The data shown in Table 2 also indicate that residues 85 and 91 of mSPEX are part of the epitope bound by antibody PJ19.1 which antibody is demonstrated herein to inhibit transplant rejection. In a preferred embodiment an antibody that binds the extracellular domain of a SPEX polypeptide inhibits transplant rejection. In another preferred embodiment, an antibody that immunoreacts with an epitope that includes residues 85 and 91 in a mSPEX polypeptide (or a corresponding residue in a hSPEX polypeptide) inhibits transplant rejection. In still another preferred embodiment, an antibody that immunoreacts with an Ig like domain of a SPEX polypeptide inhibits transplant rejection. In a further preferred embodiment, an antibody that immunoreacts with an Ig like domain of a SPEX polypeptide (e.g., SEQ ID NO:3, 45, or 88 (most preferably SEQ ID NO:3)), but does not immunoreact with a non-Ig like domain of the SPEX polypeptide (e.g., SEQ ID NO: 1, 2, 4, 5, 6, 7, 8, 9, 10, 11, 43, 44, 46, 47, 48, 49, 50, 51, 52, or 53 (most preferably SEQ ID NO: 1, 2, or 4)) inhibits transplant rejection. In still a further preferred embodiment, an antibody that immunoreacts with an amino acid sequence consisting essentially of a SPEX Ig like domain (e.g., consisting essentially of SEQ ID NO:3, 45, or 88 (most preferably SEQ ID NO:3)), inhibits transplant rejection. In yet another preferred embodiment, an antibody that includes an epitope binding pattern of PJ19.1 inhibits transplant rejection.

Although the invention has been described with reference to the above examples, it will be understood that modifications and variations are encompassed within the spirit and scope of the invention. Accordingly, the invention is limited only by the following claims.

1) A method of inhibiting transplant rejection in a mammal in need thereof, comprising: administering a therapeutically effective amount of an antibody to the mammal, wherein said antibody immunoreacts with an extracellular domain of a spleen expressed (SPEX) polypeptide, thereby inhibiting said transplant rejection.

2) The method of claim 1, wherein the transplant is an allogeneic transplant.

3) The method of claim 1, wherein said transplant is a skin graft.

4) The method of claim 1, wherein said transplant is a tissue transplant.

5) The method of claim 1, wherein said transplant is an organ transplant.

6) The method of claim 1, wherein said transplant is a bone marrow transplant.

7) The method of claim 1, further comprising administering a second transplant rejection inhibitor.

8) The method of claim 7, wherein said second transplant rejection inhibitor comprises a cyclosporin, a rapamycin, a FK506, a corticosteriod, or an antibody that immunoreacts with an interleukin (IL)-2 receptor.

9) The method of claim 1, wherein said antibody is a monoclonal antibody or a binding fragment thereof.

10) The method of claim 1, wherein said antibody does not immunoreact with an intracellular domain of said SPEX polypeptide.

11) The method of claim 1, wherein said antibody immunoreacts with an Ig-like domain of said SPEX polypeptide.

12) The method of claim 11, wherein said antibody does not immunoreact with an intracellular domain of said SPEX polypeptide.

13) The method of claim 1, wherein said antibody immunoreacts with SEQ ID NOs:3, 12, 13, 45, 54, 55, 85, 86, or 88.

14) The method of claim 13, wherein said antibody does not immunoreact with SEQ ID NOs:17, 18, 59, or 60.

15) The method of claim 1, wherein said mammal is a mouse.

16) The method of claim 1, wherein said mammal is a human.

17) The method of claim 1, wherein said mammal is a human and said anti-SPEX antibody is a humanized antibody.

18) The method of claim 1, wherein said extracellular domain of said SPEX polypeptide comprises six or more consecutive amino acids of a polypeptide consisting essentially of SEQ ID NO:55.

19) A method of decreasing an expression of a spleen expressed (SPEX) polypeptide on a lymphocyte which expresses said SPEX polypeptide, comprising contacting said lymphocyte with an anti-SPEX antibody that immunoreacts with said SPEX polypeptide, thereby decreasing said expression of said SPEX polypeptide on said lymphocyte.

20) The method of claim 19, wherein said anti-SPEX antibody is a monoclonal antibody.

21) The method of claim 19, wherein said anti-SPEX antibody specifically immunoreacts with an extracellular domain of the SPEX polypeptide.

22) The method of claim 21, wherein said anti-SPEX antibody does not immunoreact with an intracellular domain of said SPEX polypeptide.

23) The method of claim 19, wherein said anti-SPEX antibody immunoreacts with an Ig-like domain of a SPEX polypeptide.

24) The method of claim 23, wherein said anti-SPEX antibody does not immunoreact with an intracellular domain of said SPEX polypeptide.

25) The method of claim 19, wherein said anti-SPEX antibody immunoreacts with SEQ ID NOs:3, 12, 13, 45, 54, 55, 85, 86, or 88.

26) The method of claim 19, wherein said anti-SPEX antibody does not immunoreact with SEQ ID NOs:17, 18, 59, or 60.

27) A method of identifying an antibody that inhibits a transplant rejection in a mammal in need thereof, comprising:
a) providing an amino acid sequence comprising an epitope of an extracellular domain of a spleen expressed (SPEX) polypeptide, wherein said epitope is at least six amino acids in length;
b) producing antibodies to said amino acid sequence; and
c) screening said antibodies in a model of transplant rejection, thereby identifying said antibody that inhibits said transplant rejection.

28) The method of claim 27, wherein said mammal model of transplant rejection comprises an allogeneic skin graft in a murine recipient.

29) The method of claim 27, wherein said extracellular domain of said SPEX polypeptide comprises SEQ ID NOs:3, 12, 13, 45, 54, 55, 85, 86, or 88.

30) The method of claim 27, wherein said extracellular domain of said SPEX polypeptide comprises SEQ ID NO:3.

31) The method of claim 27, wherein said extracellular domain of said SPEX polypeptide comprises SEQ ID NO:13.

32) A method of inhibiting transplant rejection in a mammal in need thereof, comprising: administering a therapeutically effective amount of an antibody to the mammal, wherein said antibody immunoreacts with an amino acid sequence consisting essentially of an extracellular domain of a spleen expressed (SPEX) polypeptide, thereby inhibiting said transplant rejection.

33) A method of inhibiting transplant rejection in a mammal in need thereof, comprising: administering a therapeutically effective amount of an antibody to the mammal, wherein said antibody immunoreacts with an amino acid sequence consisting essentially of SEQ ID NO:12, thereby inhibiting said transplant rejection.

34) A method of inhibiting transplant rejection in a mammal in need thereof, comprising: administering a therapeutically effective amount of an antibody to the mammal, wherein said antibody immunoreacts with an amino acid sequence consisting essentially of an immunoglobulin like domain of a spleen expressed (SPEX) polypeptide, thereby inhibiting said transplant rejection.

35) A method of inhibiting transplant rejection in a mammal in need thereof, comprising: administering a therapeutically effective amount of an antibody to the mammal, wherein said antibody immunoreacts with an amino acid sequence consisting essentially of SEQ ID NO:3, thereby inhibiting said transplant rejection.
